(19) United States
(12) Patent Application Publication
(10) Pub. No.: US 2019/0133699 A1
(43) Pub. Date: May 9, 2019

(54) SURGICAL INSTRUMENT FOR MANUAL AND ROBOTIC-ASSISTED USE

(71) Applicant: TransEnterix Surgical, Inc., Morrisville, NC (US)

(72) Inventor: Stefano Pomati, Meda (IT)

(21) Appl. No.: 16/236,621

(22) Filed: Dec. 30, 2018

Related U.S. Application Data
(60) Provisional application No. 62/512,542, filed on May 30, 2017.

(51) Int. Cl. A61B 34/30

(52) U.S. Cl. CPC A61B 34/30; A61B 2034/302

(57) ABSTRACT

A surgical system comprises a robotic manipulator, a manually-operable instrument handle, and a surgical instrument that is removably mountable to the robotic manipulator for electromechanical movement of the instrument by the robotic manipulator, and that is removably mountable to the manually-operable instrument handle for manual movement of the instrument by a user.

### BACKGROUND

There are various types of surgical robotic systems on the market or under development. Some surgical robotic systems use a plurality of robotic arms. Each arm carries either a surgical instrument or a camera used to capture images from within the body for display on a monitor. See U.S. Pat. No. 9,358,682 and US 20160058513, which are incorporated herein by reference. Each of these types of robotic systems uses motors to position and/or orient the camera and instruments and to, where applicable, actuate the instruments. Typical configurations allow two or three instruments and the camera to be supported and manipulated by the system. Input to the system is generated based on input from a surgeon positioned at a master console, typically using input devices such as input handles and a foot pedal. Motion and actuation of the surgical instruments and the camera is controlled based on the user input. The image captured by the camera is shown on a display at the surgeon console. The console may be located patient-side, within the sterile field, or outside of the sterile field.

It is common that robotic surgical systems include surgical device assemblies that are removably mounted to the manipulator arms. This allows the users to remove and replace the instrument mounted to any given manipulator during the course of a surgical procedure. Thus multiple types of instruments (e.g. graspers, dissectors, scissors, needle holders, clip appliers, mono-polar or bipolar instruments) are often available to be engaged with the robotic manipulator and may be interchanged during the course of a procedure. The shaft length and diameter can vary. As non-limiting examples, instruments might be provided in diameters of 3, 5 and 10 mm, and in lengths of 310 and 410 mm.

It is also common to have manual surgical instruments on hand during a surgical procedure, in case the surgeon elects perform certain steps manually, or to convert the surgery to a manual procedure. This application describes instruments that can be both mounted to a robotic manipulator arm for robotic-assisted use, and that can be mounted to an instrument handle for manual use.

### BRIEF DESCRIPTION OF THE DRAWINGS

FIG. 1 schematically illustrates a robotic manipulator of a type used in robotic surgical procedures.

FIG. 2A is a top plan view of a surgical device assembly.

FIG. 2B illustrates the step of mounting the surgical device assembly onto the manipulator of FIG. 1.

FIG. 3 shows a side elevation view of a surgical instrument, proximal housing, and manual handle and illustrates interchangeably of the handle with the proximal housing of the surgical device.

### DETAILED DESCRIPTION

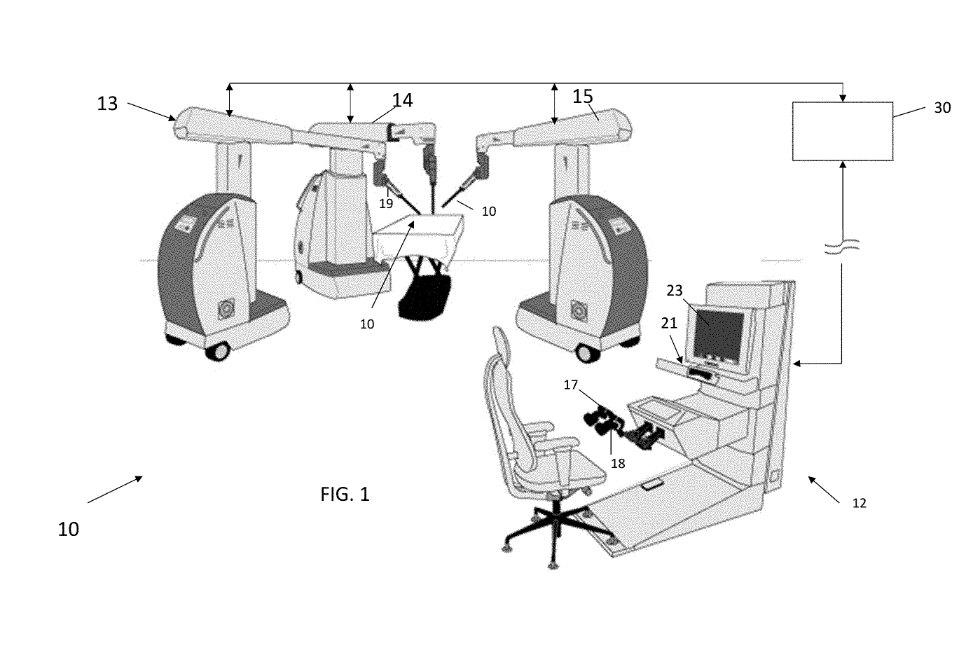

FIG. 1 shows components of a robotic surgical system 10 of the type described in U.S. Pat. No. 9,358,682 and US 20160058513. Features of the system 10 are shown to facilitate an understanding of the way in which the concepts of the present invention may be implemented, but it should be understood that the invention may be used with a variety of different surgical or industrial robotic systems and is not limited to use with system 10.

System 10 comprises at least one robotic manipulator arm 13-15 which operates under the control of a command console or cockpit 12 operated by the surgeon. The robotic manipulator (or each robotic manipulator) has an effector unit 20 with a terminal portion designed to support, position and orient a surgical device assembly 14. The surgical device assembly includes a surgical instrument having shaft 15 and a distal end effector 17 positionable within a patient 16.

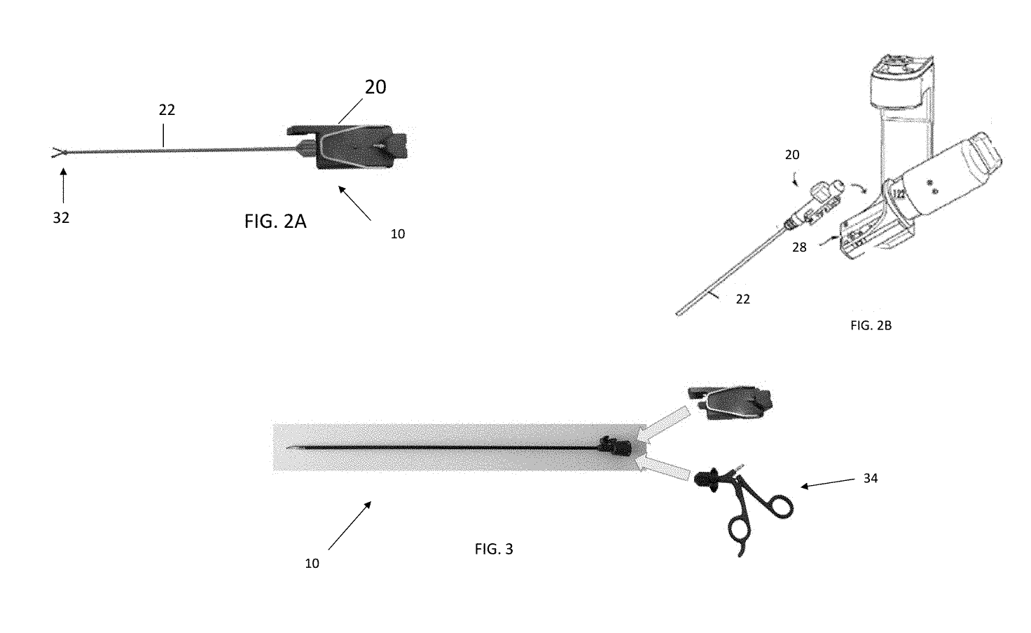

The surgical device assembly (FIG. 2A) includes an instrument shaft 22 extending from proximal part that is received by the terminal portion 28 (FIG. 3) of the robotic manipulator. In the illustrated configuration, the proximal part of the surgical device assembly is a proximal housing 20 that is received by, and mechanically engages with, the terminal portion 28 as shown in FIG. 2B. The proximal housing 20 may be removably attached to the instrument shaft 15 to facilitate cleaning and sterilization of these components.

An end effector 32 is positioned on the distal end of the shaft 22. The end effector 32 of the surgical device assembly may be one of many different types of that are used in surgery including, without limitation, end effectors having one or more of the following features: jaws that open and close, joints that articulate in one or more degrees of freedom, distal shaft sections that bend, a tip that rolls axially relative to the shaft 22, a tip mounted on a shaft that rolls axially relative to the corresponding robotic manipulator arm.

The system includes instrument actuators for driving the motion of the end effector 32. These actuators, which might be motors or other types of actuators (e.g. hydraulic/pneumatic), are positioned in the terminal portion 28 of the robotic manipulator, or in the housing 20 of the surgical device assembly, or some combination of the two. In the latter example, some motion of the end effector might be driven using one or more motors in the terminal portion 28 of the robotic manipulator, while other motion might be driven using motors in the housing 20. During use, the robotic system controls movement of the robotic manipulator and movement of the end effector (e.g. jaw open/close, tip roll, articulating or bending, etc.) based on surgeon input received by the system via the console 12. The control signals used to generate the various types of movement depend in some cases on the geometry, length, weight, or other parameters of the surgical instrument 10.

At times during a surgical procedure, a member of the surgical staff may wish to perform steps of the procedure manually rather than using the robotic manipulators. A set of manual laparoscopic/minimally invasive instruments is thus typically available in the surgical procedure room for easy access. If a user wishes to use one of these manual instruments in place of a robotically manipulated one, the robotically manipulated instrument is removed from the patient and replaced with the manual one.

The present application is directed to a system that allows the user to detach a surgical instrument from the robotic manipulator and mount a manual handle to the surgical instrument, allowing an instrument that was being manipulated by the robotic system to be operated manually by the surgeon. This can avoid the need to remove the instrument from the body before manual steps can be performed.

The system includes a surgical instrument having a shaft 22 as described above. The surgical instrument includes a proximal housing 20 which may removable from the shaft. The proximal housing 20 is mountable to the terminal portion 28 of a robotic manipulator. As discussed above, the proximal housing 20 has mechanical features that are actuated by corresponding features in the terminal portion 28 in order to actuate certain functions of the instrument, such as the opening/closing of the instrument's jaws and/or bending or articulation of the instrument shaft or end effector.

The system further includes a manually operable instrument handle 34 operatively engageable to the instrument.

In one embodiment, the handle is configured to couple to the shaft 22 of the surgical instrument 10. The handle includes manually moveable actuation elements (e.g. knobs, triggers, sliders etc) of the type known in the art that operably engage corresponding actuation elements of the surgical instrument for manual actuation the functions of the instrument. These manually moveable actuation elements are operatively coupled to actuation elements of the instrument that are moved to cause the desired instrument function (e.g. jaw actuation, bending/articulation). Thus, a single instrument 10 may be attached to the handle 34 to allow the surgeon to manually control the instrument's functions for manual use, and the same instrument 10 may be coupled to its proximal housing 20 and attached to the robotic manipulator as described above when robotic use of that instrument is desired. In other words, when the instrument is mounted to the robotic manipulator, the motors of the manipulator arm control movement and positioning of the surgical instrument within the body cavity, and motors in arm (including, without limitation, the terminal portion 28) control functions at the instrument tip 32 and/or shaft such as jaw open/close, tip roll, articulating or bending, etc. When the instrument is mounted to the handle, all such functions are performed manually.

In a second embodiment, the handle is are similar to those described above, except that they are configured to operably couple to the removable proximal housing 20 of the surgical instrument. With this configuration, a single instrument 10 and proximal housing assembly 20 may be attached to the handle to allow the surgeon to manually control the instrument's functions (e.g. jaw actuation, bending/articulation) for manual use, and the same instrument 10 and proximal housing assembly 20 may be attached to the robotic manipulator as described above when robotic use of that instrument is desired.

A third embodiment is similar to the second embodiment, except that each instrument has a first removably attachable proximal housing 20 that is for use with the manual handle, and a second removably attachable proximal housing that engages with the terminal portion of the robotic manipulator for robotic use.

The handles may include features allowing them to be used with electrosurgical instruments. For example, a handle might include a connector allowing it to be coupled to a source of monopolar or bipolar energy when used with a monopolar or bipolar instrument.

All patents and applications referred to herein, including for purposes of priority, are incorporated herein by reference.

What is claimed is:

1. A surgical system comprising:
a robotic manipulator;
a manually-operable instrument handle;
a surgical instrument that is removably mountable to the robotic manipulator for electromechanical movement of the instrument by the robotic manipulator, and that is removably mountable to the manually-operable instrument handle for manual movement of the instrument by a user.

2. The surgical system of claim 1, wherein the instrument includes a proximal housing, the proximal housing including first actuation elements operatively engageable to corresponding second actuation elements of the robotic manipulator such that electromechanical motion of the second actuation elements causes actuation of an end effector of the instrument.

3. The surgical instrument of claim 2 wherein the handle includes third actuation elements, the third actuation elements operably coupleable to the first actuation elements of the instrument such that manual-controlled motion of the third actuation elements causes actuation of the end effector of the instrument.

4. The surgical instrument of claim 3, wherein the proximal housing is removably attachable to the instrument and wherein the handle is removably attachable to the instrument when the proximal housing is not attached to the instrument.

5. The surgical instrument of claim 3, wherein the handle is removably attachable to the instrument when the proximal housing is attached to the instrument.

6. A surgical method comprising:
positioning a surgical instrument on a robotic manipulator;
positioning a distal end of the surgical instrument in a body cavity;
performing a surgical step within the body cavity using the surgical instrument, the surgical instrument distal end moveable within the body cavity by the robotic manipulator;
detaching the surgical instrument from the robotic manipulator and coupling a handle to the surgical instrument;
performing a surgical step within the body cavity using the surgical instrument wherein the surgical instrument distal end is moved within the body cavity by manual operation of the handle by a hand of a user.

7. The method of claim 6, wherein the step of detaching the surgical instrument from the robotic manipulator and coupling the handle to the surgical instrument is performed without removing the distal end of the instrument from the body cavity.

8. The method of claim 6, wherein:
the surgical instrument includes a proximal housing;
the step of positioning the surgical instrument on the robotic manipulator includes operably coupling first actuation elements of the proximal housing with second actuation elements of the robotic manipulator.

9. The method of claim 8 wherein the method includes detaching the proximal housing from the surgical instrument prior to coupling the handle to the surgical instrument, the handle including third actuation elements, the third actuation elements operably coupleable to fourth actuation elements of the instrument such that manual-controlled motion of the third actuation elements causes actuation of the end effector of the instrument.

10. The method of claim 8, wherein the step of coupling the handle to the surgical instrument includes the step of coupling the handle to the proximal housing, the handle including third actuation elements, the third actuation elements operably coupleable to the first actuation elements of the proximal housing such that manual-controlled motion of the third actuation elements causes actuation of the end effector of the instrument.
